(19) United States
(12) Patent Application Publication
(10) Pub. No.: US 2023/0133446 A1
(43) Pub. Date: May 4, 2023

(54) METHODS FOR PROVIDING A SOLID HMO PRODUCT, AND SOLID HMO PRODUCTS OBTAINED THEREBY

(71) Applicant: GLYCOM A/S, Hørsholm (DK)

(72) Inventor: Pierre CHASSAGNE, Beaumont (FR)

(21) Appl. No.: 17/906,701

(22) PCT Filed: Jan. 29, 2021
(86) PCT No.: PCT/IB2021/050703

§ 371 Date: Sep. 19, 2022

(30) Foreign Application Priority Data
Mar. 20, 2020 (DK) PA 2020 70175

(51) Int. Cl. A23C 9/20; C07H 3/06; C12P 19/18; F26B 5/12; F26B 15/18; A23C 1/03; A23L 33/125; A23L 3/48; A23P 10/22; A23L 33/00; A23C 9/146; A23C 9/142

(52) U.S. Cl. CPC A23C 9/203; C07H 3/06; C12P 19/18; F26B 5/12; F26B 15/18; A23C 1/03; A23L 33/125; A23L 3/485; A23P 10/22; A23L 33/40; A23C 9/146; A23C 9/142

(57) ABSTRACT

Various methods are provided for providing a solid HMO product, said methods including drum-drying, belt drying, preferably vacuum bed drying, or granulation. Solid HMO products are obtained via said methods, as well as solid HMO products per se. The methods can provide solid HMO products which are different to those achieved via other methods.

### TECHNICAL FIELD

Various methods for providing a solid HMO product are provided, as well as solid HMO products obtained via such methods, and novel solid HMO products themselves.

### BACKGROUND

Human milk oligosaccharides (HMOs) are a heterogeneous mixture of soluble glycans found in human milk. They are the third most abundant solid component after lactose and lipids in human milk and are present in concentrations of 5-25 g/l (Bode: Human milk oligosaccharides and their beneficial effects, in: Handbook of dietary and nutritional aspects of human breast milk (Zibadi et al., eds.), pp. 515-31, Wageningen Academic Publishers (2013)).

Human milk oligosaccharides (HMOs) have become of great interest in the past few years due to their important functions in human development. To date, the structures of at least 115 HMOs have been determined (see Urashima et al.: Milk Oligosaccharides, Nova Biomedical Books, New York, 2011, ISBN: 978-1-61122-831-1; Chen Adv. Carbohydr. Chem. Biochem. 72, 113 (2015)), and considerably more are probably present in human milk. The thirteen core structures identified to date, for the at least 115 HMOs, are listed in Table 1:

TABLE 1
Core HMO structures

| No | Core name | Core structure |
|---|---|---|
| 1 | lactose (Lac) | Galβ1-4Glc |
| 2 | lacto-N-tetraose (LNT) | Galβ1-3GlcNAcβ1-3Galβ1-4Glc |
| 3 | lacto-N-neotetraose (LNnT) | Galβ1-4GlcNAcβ1-3Galβ1-4Glc |
| 4 | lacto-N-hexaose (LNH) | Galβ1-3GlcNAcβ1-3(Galβ1-4GlcNAcβ1-6)Galβ1-4Glc |
| 5 | lacto-N-neohexaose (LNnH) | Galβ1-4GlcNAcβ1-3(Galβ1-4GlcNAcβ1-6)Galβ1-4Glc |
| 6 | para-lacto-N-hexaose (para-LNH) | Galβ1-3GlcNAcβ1-3Galβ1-4GlcNAcβ1-3Galβ1-4Glc |
| 7 | para-lacto-N-neohexaose (para-LNnH) | Galβ1-4GlcNAcβ1-3Galβ1-4GlcNAcβ1-3Galβ1-4Glc |
| 8 | lacto-N-octaose (LNO) | Galβ1-3GlcNAcβ1-3(Galβ1-4GlcNAcβ1-3Galβ1- |
| | | 4GlcNAcβ1-6)Galβ1-4Glc |
| 9 | lacto-N-neooctaose (LNnO) | Galβ1-4GlcNAcβ1-3(Galβ1-3GlcNAcβ1-3Galβ1- |
| | | 4GlcNAcβ1-6)Galβ1-4Glc |
| 10 | iso-lacto-N-octaose (iso-LNO) | Galβ1-3GlcNAcβ1-3(Galβ1-3GlcNAcβ1-3Galβ1- |
| | | 4GlcNAcβ1-6)Galβ1-4Glc |
| 11 | para-lacto-N-octaose (para-LNO) | Galβ1-3GlcNAcβ1-3Galβ1-4GlcNAcβ1-3Galβ1- |
| | | 4GlcNAcβ1-3Galβ1-4Glc |
| 12 | lacto-N-neodecaose (LNnD) | Galβ1-3GlcNAcβ1-3[Galβ1-4GlcNAcβ1-3(Galβ1- |
| | | 4GlcNAcβ1-6)Galβ1-4GlcNAcβ1-6]Galβ1-4Glc |
| 13 | lacto-N-decaose (LND) | Galβ1-3GlcNAcβ1-3[Galβ1-3GlcNAcβ1-3(Galβ1- |
| | | 4GlcNAcβ1-6)Galβ1-4GlcNAcβ1-6]Galβ1-4Glc |

Low cost ways have been sought for making industrial quantities of as many as possible of the HMOs, so that their uses in nutritional and therapeutic formulations for infants, as well as possibly children and adults, could be discovered, developed and exploited by researchers worldwide. A few HMOs have recently been chemically synthesized in high yields, while other methods have used fermentation techniques using cultured microorganisms. WO 2013/185780 and WO 2019/110800 disclose spray-drying of HMOs and mixtures of HMOs.

Certain crystalline HMOs have shown to be relatively unstable when stored for extended periods without refrigeration. They have tended to melt and thereby become sticky and form agglomerations.

Accordingly, it is also an object of the present invention to provide a method for providing solid HMO products with enhanced stability, so that they can be stored for extended periods without refrigeration, for example at temperatures of up to 30° C. or even higher, preferably up to 40° C. or even higher.

### SUMMARY

In a first aspect the present invention relates to a method for providing a solid HMO product comprising one or more synthetic human milk oligosaccharides (HMOs), HMO precursors and/or by-products of HMO synthesis, said process comprising a step of drum-drying an aqueous composition comprising said one or more human milk oligosaccharides (HMOs), HMO precursors and/or by-products of the HMO synthesis.

Preferably, the aqueous composition comprises one or more HMOs and optionally HMO precursors and/or by-products of HMO synthesis, and method thereby provides the solid HMO product comprising one or more HMOs that optionally comprises HMO precursors and/or by-products of the HMO synthesis.

In a second aspect, the present invention relates to a method for providing a solid HMO product comprising one or more synthetic human milk oligosaccharides (HMOs), HMO precursors and/or by-products of HMO synthesis, said process comprising a step of belt drying, preferably vacuum belt drying, an aqueous composition comprising said one or more human milk oligosaccharides (HMOs), HMO precursors and/or by-products of the HMO synthesis.

Preferably, the aqueous composition comprises one or more HMOs and optionally HMO precursors and/or by-products of HMO synthesis, and method thereby provides the solid HMO product comprising one or more HMOs that optionally comprises HMO precursors and/or by-products of the HMO synthesis.

In a third aspect, the present invention relates to a method for providing a solid HMO product comprising one or more synthetic human milk oligosaccharides (HMOs), HMO precursors and/or by-products of HMO synthesis, said process comprising a step of granulating an aqueous composition comprising said one or more human milk oligosaccharides (HMOs), HMO precursors and/or by-products of the HMO synthesis to form a granulated composition, followed by a step of drying said granulated composition.

Preferably, the aqueous composition comprises one or more HMOs and optionally HMO precursors and/or by-products of HMO synthesis, and method thereby provides the solid HMO product comprising one or more HMOs that optionally comprises HMO precursors and/or by-products of the HMO synthesis after drying the granulated composition.

Furthermore, a solid HMO product comprising one or more synthetic human milk oligosaccharides (HMOs) is provided, obtained by the methods described herein.

A drum-dried, a belt dried, preferably vacuum belt dried, or a granulated solid HMO product comprising one or more synthetic human milk oligosaccharides (HMOs) is also provided, obtained by the methods described herein.

Also provided is a solid HMO product comprising one or more human milk oligosaccharides (HMOs), said solid HMO product having a glass transition temperature (T_{g}) more than 100° C., such as from around 105° C. to around 160° C., e.g. around 100-150° C. or around 120-140° C. The term “around” in this context means a deviation from the indicated value of +/−2.5° C.

A food product, in particular a nutritional formulation, e.g. an infant nutritional formulation, is also provided, which comprises a solid HMO product as described herein.

Further features of the invention are evident from the following description, numbered aspects, examples and claims.

### FIGURES

FIG. 1 shows the XRPD spectrum of the material obtained in example 3.

### DETAILED DISCLOSURE

Unless otherwise defined, the term “around” is to be defined as a value that deviates from 0.1 to 5% from the indicated value, such as 0.1-2.5% or 0.1-1.0%.

As above, the present technology provides three methods for providing a solid HMO product. The solid HMO product comprises one or more synthetic human milk oligosaccharides (HMOs), HMO precursors and/or by-products of HMO synthesis. Preferably, the solid HMO product comprises one or more synthetic human milk oligosaccharides (HMOs), and is obtained via drying (by various means) an aqueous composition comprising said one or more human milk oligosaccharides (HMOs).

The first method comprises a step of drum-drying an aqueous composition comprising said one or more human milk oligosaccharides (HMOs), HMO precursors and/or by-products of the HMO synthesis. Solid HMO product is thus obtained.

The second method comprises a step of belt drying, preferably vacuum belt drying, an aqueous composition comprising said one or more human milk oligosaccharides (HMOs), HMO precursors and/or by-products of the HMO synthesis. Solid HMO product is thus obtained.

The third method comprises a step of granulating an aqueous composition comprising said one or more human milk oligosaccharides (HMOs), HMO precursors and/or by-products of the HMO synthesis to form a granulated composition, followed by a step of drying said granulated composition. Solid HMO product is thus obtained.

All methods described herein use an aqueous composition comprising the one or more human milk oligosaccharides (HMOs), HMO precursors and/or by-products of the HMO synthesis. The aqueous composition may be an aqueous solution or an aqueous suspension of the human milk oligosaccharides (HMOs), HMO precursors and/or by-products of the HMO synthesis, and is preferably an aqueous solution of said one or more synthetic human milk oligosaccharides (HMOs), HMO precursors and/or by-products of HMO synthesis.

The aqueous composition suitably comprises two or more HMOs, such as e.g. three or more HMOs, or four or more HMOs. The aqueous composition may comprise even 5 or more HMOs. The HMOs may be selected from the group consisting of LNT, LNnT, 2′-FL, 3-FL, DFL, LNFP I, 3′-SL, 6′-SL, FSL, LST a, LST b, and DS-LNT.

The aqueous composition may include additional components such as e.g. salts, pH regulating agents or solubilising agents. The pH of the aqueous composition is suitably between 3-7, preferably between 4-6.

In each case, the drying step suitably takes place to a moisture content in the solid HMO product of below 15%, preferably below 10%, more preferably below 7%, most preferably below 6%.

In one particular aspect, the one or more synthetic HMOs are produced by means of fermentation, prior to said drying step.

Suitably, according to this aspect, one or more purification steps are carried out on the HMO fermentation broth, enzymatic reaction milieu or synthetic reaction mixture, prior to said drying step. The purification steps carried out on the fermentation broth, enzymatic reaction milieu or synthetic reaction mixture may be selected from one or more of:
removing solid material e.g. from the HMO synthesis milieu, such as fermentation material (such as proteins and DNA) or enzymatic reaction material (proteins);
removing salts and charged molecules (such as small DNA fragments, organic acids, peptides);
removing uncharged or non-charged material (e.g. lipids, polysaccharides e.g. endotoxin, colorants from fermentation);
removing HMO precursors and/or by-products;
separating HMOs, if an HMO mixture is produced;
removing an excess of water.

In aspects, said one or more synthetic HMOs are produced by means of fermentation and the fermentation broth is further processed to prepare an aqueous composition comprising said one or more synthetic human milk oligosaccharides (HMOs).

In one embodiment, especially if the drying step is belt drying, preferably vacuum belt drying, as disclosed in the present application, the fermentation broth is treated by the following steps prior to drying:
a) clarifying the broth to remove suspended particulates and contaminants, particularly cells, cell components, insoluble metabolites and debris from a fermentation process; then
b) removing substantially all the proteins, as well as peptides, amino acids, RNA and DNA and any endotoxins and glycolipids that could interfere with the subsequent purification step, from the aqueous solution obtained in step a).

In step a), the broth is clarified in a conventional manner, e.g. by centrifugation or filtration. Preferably the aqueous medium is first flocculated and then centrifuged or filtered to remove any remaining insoluble particulates and contaminants, as well as cells and cell components and insoluble metabolites and debris.

In step b), proteins and related impurities are removed from the aqueous medium obtained previously in a conventional manner, e.g. by ultrafiltration, nanofiltration, tangential flow high-performance filtration, tangential flow ultrafiltration, affinity chromatography, ion exchange chromatography, hydrophobic interaction chromatography, gel filtration, size exclusion chromatography and/or active charcoal treatment. The active charcoal treatment helps to remove or at least reduce the amount of colorizing agents and/or water soluble contaminants, such as salts, if required. Ion exchange chromatography efficiently removes charged components such as salts, colour bodies, proteins, amino acids, lipids and DNAs.

Accordingly, the method my further comprise the following separation/purification steps of the fermentation broth in any order prior to drying, preferably belt drying, more preferably vacuum belt drying:
i) ultrafiltration (UF),
ii) nanofiltration (NF), and
iii) treatment with an ion exchange resin.

Advantageously, step i) is conducted before step ii). More advantageously, the step i) is conducted before any of the steps ii) and iii). Preferably, the method is performed in the order where step ii) follows step i) and step iii) follows step ii). The method may further comprise an active charcoal treatment after UF, NF or ion exchange resin treatment.

In one aspect of interest, the one or more synthetic HMOs are produced by means of fermentation, and the fermentation broth is fed directly to the drum dryer as the aqueous composition comprising said one or more synthetic human milk oligosaccharides (HMOs), without any intermediate purification steps.

The HMO content of the aqueous composition, and whether a solution or slurry is formed, depends the solubility of the HMO in question.

Typically for 2′-FL, the minimum concentration is estimated at Brix 50. Preferably, the process will be run on a saturated solution (ex: Brix 58-65), it but can also be imagined to be run on a supersaturated metastable solution or an aqueous slurry (up to 85% dry matter).

### Synthetic Human Milk Oligosaccharides (HMOs)

The human milk oligosaccharides referred to in the present technology are synthetic, i.e. produced by chemical or biochemical processes in vitro or in vivo. The synthetic HMOs used in the present methods and products may be selected from one or more of LNT, LNnT, 2′-FL, 3-FL, DFL, LNFP I, 3′-SL, 6′-SL, FSL, LST a, LST b, and DS-LNT, preferably LNT, LNnT, 2′-FL, 3-FL, DFL, LNFP I, 3′-SL and 6′-SL.

The synthetic HMOs may be neutral or acidic (sialylated).

The term “neutral human milk oligosaccharide” means a non-sialylated (therefore neutral) complex carbohydrate found in human breast milk (Urashima et al.: Milk oligosaccharides, Nova Biomedical Books, 2011; Chen Adv. Carbohydr. Chem. Biochem. 72, 113 (2015)) comprising a core structure being a lactose unit at the reducing end that is a) substituted with one or two α-L-fucopyranosyl moieties, b) substituted with a galactosyl residue, or c) elongated, via its 3′-OH group, by an N-acetylglucosamine, a lacto-N-biose (Galβ1-3GlcNAc) or an N-acetyllactosamine (Galβ1-4GlcNAc) moiety. The N-acetyllactosamine containing derivatives can be further substituted with N-acetyllactosamine and/or lacto-N-biose (lacto-N-biose is always a non-reducing terminal). The N-acetyllactosamine and the lacto-N-biose containing derivatives can optionally be substituted by one or more α-L-fucopyranosyl moieties.

Examples of neutral trisaccharide HMOs include 2′-O-fucosyllactose (2′-FL, Fucα1-2Galβ1-4Glc), 3-O-fucosyllactose (3-FL, Galβ1-4(Fucα1-3)Glc) or lacto-N-triose II (GlcNAcβ1-3Galβ1-4Glc); examples of neutral tetrasaccharide HMOs include 2′,3-di-O-fucosyllactose (DFL, Fucα1-2Galβ1-4(Fucα1-3)Glc), lacto-N-tetraose (LNT, Galβ1-3GlcNAcβ1-3Galβ1-4Glc) or lacto-N-neotetraose (LNnT, Galβ1-4GlcNAcβ1-3Galβ1-4Glc); examples of neutral pentasaccharide HMOs include lacto-N-fucopentaose I (LNFP I, Fucα1-2Galβ1-3GlcNAcβ1-3Galβ1-4Glc), lacto-N-fucopentaose II (LNFP II, Galβ1-3(Fucα1-4)GlcNAcβ1-3Galβ1-4Glc), lacto-N-fucopentaose III (LNFP III, Galβ1-4(Fucα1-3)GlcNAcβ1-3Galβ1-4Glc), lacto-N-fucopentaose V (LNFP V, Galβ1-3GlcNAcβ1-3Galβ1-4(Fucα1-3)Glc), lacto-N-fucopentaose VI (LNFP VI, Galβ1-4GlcNAcβ1-3Galβ1-4(Fucα1-3)Glc); examples of neutral hexasaccharide HMOs include lacto-N-difucohexaose I (LNDFH I, Fucα1-2Galβ1-3(Fucα1-4)GlcNAcβ1-3Galβ1-4Glc), lacto-N-difucohexaose II (LNDFH II, Galβ1-3(Fucα1-4)GlcNAcβ1-3Galβ1-4(Fucα1-3)Glc), lacto-N-difucohexaose III (LNDFH III, Galβ1-4(Fucα1-3)GlcNAcβ1-3Galβ1-4(Fucα1-3)Glc), lacto-N-hexaose (LNH, Galβ1-3GlcNAcβ1-3(Galβ1-4GlcNAcβ1-6)Galβ1-4Glc), para-lacto-N-hexaose (pLNH, Galβ1-3GlcNAcβ1-3Galβ1-4GlcNAcβ1-3Galβ1-4Glc), lacto-N-neohexaose (LNnH, Galβ1-4GlcNAcβ1-3(Galβ1-4GlcNAcβ1-6)Galβ1-4Glc) or para-lacto-N-neohexaose (pLNnH, Galβ1-4GlcNAcβ1-3Galβ1-4GlcNAcβ1-3Galβ1-4Glc).

The term “sialylated human milk oligosaccharide” means a sialylated complex carbohydrate found in human breast milk (Urashima et al.: Milk oligosaccharides, Nova Biomedical Books, 2011; Chen Adv. Carbohydr. Chem. Biochem. 72, 113 (2015)) comprising a core structure being a lactose unit at the reducing end that can be elongated by one or more β-N-acetyl-lactosaminyl and/or one or more β-lacto-N-biosyl units, and which core structure is substituted by an α-N-acetyl-neuraminyl (sialyl) moiety and optionally can be substituted by an α L-fucopyranosyl moiety. In this regard, the acidic HMOs have at least one sialyl residue in their structure.

Examples of acidic HMOs include 3′-sialyllactose (3′-SL), 6′-sialyllactose (6′-SL), 3-fucosyl-3′-sialyllactose (FSL), LST a, fucosyl-LST a (FLST a), LST b, fucosyl-LST b (FLST b), LST c, fucosyl-LST c (FLST c), sialyl-LNH (SLNH), sialyl-lacto-N-hexaose (SLNH), sialyl-lacto-N-neohexaose I (SLNH-I), sialyl-lacto-N-neohexaose II (SLNH-II) and disialyl-lacto-N-tetraose (DS-LNT).

Different mixtures of HMOs produced enzymatically are described in earlier patent applications WO 2016/199071, WO 2017/221208, WO 2017/103850, WO 2016/157108, WO 2016/063262, which are incorporated by reference.

Any artificial blends of HMOs synthesised by any available methods are also included in the scope of the present invention.

### HMO Precursors

The term “HMO precursor” in the present context refers to a compound being involved in the biosynthetic pathway of one or more HMOs according to the invention, which are produced and naturally present in the host cell or imported into the cell from the extracellular medium. Some non-limiting examples of HMO precursors are listed below:

| Precursor: | Product: |
|---|---|
| UDP-GlcNAc | LNT, LNnT, LNFP-I, LNFP-II, LNFP-III, LNFP-V, LNFP-VI, LNDFH-I, |
| | LNDFH-II, pLNH, F-pLNH I, 3′-SL, 6′-SL, pLNnH, (F)LST a, |
| | (F)LST b, (F)LST c, (F)LST d |
| UDP-Gal | LNT, LNnT, LNFP-I, LNFP-II, LNFP-III, LNFP-V, LNFP-VI, LNDFH-I, |
| | LNDFH-II, pLNH, F-pLNH I, pLNnH, LST a, LST b, LST c, LST d |
| GDP-fucose | LNFP-I, LNFP-II, LNFP-III, LNFP-V, LNFP-VI, LNDFH-I, LNDFH-II, |
| | F-pLNH I, 2′-FL, 3-FL, DFL, FLST a, FLST b, FLST c, FLST d |

Precursors also include lactose, fucose, sialic acid and derivatives thereof. In some embodiments, a precursor may be a by-product of an HMO fermentation.

### By-Products of the HMO Synthesis

By the term “by-products” is meant carbohydrate or carbohydrate-like structures that are formed during fermentation beside the HMO of interest. For example, in fermentation of 2′-FL, fucosylated carbohydrates other than 2′-FL, e.g. DFL, can be found as by-products in high-titre aqueous fermentation broths in a DFL/2′-FL ratio of 2:8 to 1:9 by weight. The fucosylated carbohydrates other than 2′-FL, in the aqueous solution, can be any other monofucosylated lactoses that can be formed during fermentation as a result of a deficient, defective or impaired fucosylation other than an α-1,2-fucosylation on the galactose moiety of lactose (e.g. one leading to 3-O-fucosyllactose, 3-FL), or of fucose migration of 2′-FL under the cultivation condition or post-fermentative operations, or of fucose hydrolysis from multifucosylated, preferably difucosylated, lactose. Such an other fucosylated lactose can also be a multifucosylated, preferably difucosylated, lactose that can be formed as a result of overfucosylation of lactose under the cultivation condition. The difucosylated lactose is preferably 2,2′-di-O-fucosyllactose or 2′,3-di-O-fucosyllactose (DFL), particularly DFL as a characteristic by-product formed in a fermentative production of 2′-FL. This aqueous solution can also contain carbohydrate-like contaminants like 2′-O-fucosyl lactulose, lactose, lactulose, fucose, glucose, galactose and the like. These contaminants can be formed during fermentation or in post-fermentative purification/isolation steps, e.g. by rearrangement (e.g. 2′-O-fucosyl lactulose, lactulose) or by hydrolysis (e.g. fucose, glucose, galactose, lactose), or can be unconsumed educts or ingredients added during the fermentation (e.g. glucose as a carbon source). These contaminants are typically in a concentration of not more than 1-2 w/w % (individually), or 5-7 w/w % (altogether) in the aqueous solution of a desired HMO intended for drying. The aqueous solution typically can further contain up to 15 w/w %, preferably up to 10 w/w %, more preferably no more than 5 w/w %, particularly no more than 1 w/w %, of the HMO precursor, e.g. lactose as unused acceptor added to the fermentation broth.

The spectrum of fermentation by-products of other HMO structures, like sialylated or neutral non-fucosylated HMOs, could be different from the spectrum of by-products of the fucosylated HMOs fermentation, as the spectrum of precursors and by-products, in general, depends on the desired HMO structure and substrate specificity of the enzyme(s) used for the production of the HMO. Non-limiting examples of HMOs and HMO mixtures, where one HMO is a desired HMO and other HMOs are “by-products” that can be generated in fermentation of the desired HMO using some selected enzymes, are presented in Table 2 below.

TABLE 2

| Enzyme/Gene | HMO example |
|---|---|
| β-1,3-N- | LNT, LNnT, LNFP-I, LNFP-II, LNFP-III, LNFP-V, LNFP-VI, |
| acetylglucosaminyltransferase/lgtA | LNDFH-I, LNDFH-II, pLNH, F-pLNH I, pLNnH |
| β-1,4-galactosyltransferase/galT | LNnT, LNFP-III, LNFP-VI, pLNH, F-pLNH I, pLNnH |
| β-1,3-galactosyltransferase/cpsIBJ | LNT, LNFP-I, LNFP-II, LNFP-V, LNDFH-I, LNDFH-II, pLNH, |
| | F-pLNH I |
| α-1,3-fucosyltransferase/ | 2′-FL, 3-FL, DFL, LNFP-I, LNFP-III, LNFP-V, LNFP-VI, |
| MAMA_R764 | LNDFH-II, F-pLNH I |
| α-1,3-fucosyltransferase/Mg791 | 2′-FL, 3-FL, DFL, LNFP-I, LNFP-III, LNFP-V, LNFP-VI, |
| | LNDFH-II, F-pLNH I |
| α-1,3-fucosyltransferase/ | 2′-FL, 3-FL, DFL, LNFP-I, LNFP-III, LNFP-V, LNFP-VI, |
| Moumou_00703 | LNDFH-II, F-pLNH I |
| α-1,3-fucosyltransferase/futA | 2′-FL, 3-FL, DFL, LNFP-I, LNFP-III, LNFP-V, LNFP-VI, |
| | LNDFH-II, F-pLNH I |
| α-1,2-fucosyltransferase/futC | 2′-FL, DFL, LNFP-I, LNDFH-I |
| α-1,3-fucosyltransferase fucT | 2′-FL, 3-FL, DFL, LNFP-I, LNFP-III, LNFP-V, LNFP-VI, |
| | LNDFH-II, F-pLNH I |
| α-1,4-fucosyltransferase/fucTIII | LNDFH-I, LNDFH-II |
| α-1,3/4-fucosyltransferase/ | LNFP-II, LNDFH-I, LNDFH-II |
| CMP-Neu5Ac synthetase/neuA | 3′-SL, 6′-SL |
| sialic acid synthase/neuB | 3′-SL, 6′-SL |
| GlcNAc-6-phosphate-2-epimerase/ | 3′-SL, 6′-SL |
| neuC | |

In some embodiments, the terms “precursor” and “by-product” may relate to the same molecule.

### Drum Drying

Drum drying is a technique which involves depositing the aqueous composition comprising the one or more human milk oligosaccharides (HMOs), HMO precursors and/or by-products of the HMO synthesis onto a heated surface of a large drum. The drum rotates while water evaporates. A doctor blade is used to scrape dried product from the drum surface.

In one configuration, the drum dryer comprises one or more applicator rollers. The aqueous composition is deposited in the nip between applicator rollers and/or in the nip between the applicator rollers and the drying drum. There are other configurations of drum dryer without applicator rollers. For instance, drum dryers may incorporate a dip feed or a sprayer or splasher to apply the composition to the heated drum.

In one aspect, therefore, the drum-dryer used in the step of drum-drying does not comprise any applicator rollers. In another aspect, the drum-dryer used in the step of drum-drying is a double drum dryer.

A drum dryer may be single-drum (with one heated drum) or double-drum (with two heated drums, where the composition to be dried is fed into the nip between heated drums).

The present technology encompasses vacuum drum dryers, which—as they operate at reduced pressures—can advantageously avoid issues relating to high temperatures. Vacuum drum dryers typically operate at 20-60 mbars pressure, allowing the product to be exposed to low temperatures (40-120° C., preferably 60-90° C.). Besides operating at lower temperatures, vacuum drum dryers are also more sanitary (as they operate within a closed environment) and possibly more suitable for infant food production. Suitably, therefore the drum-dryer used in the step of drum-drying is a vacuum drum dryer.

Drying using a drum dryer takes place to a moisture content in the solid HMO product of below 15%, preferably below 10%, more preferably below 7%, most preferably below 6%, such as 2-3%.

The person skilled in the art is able to adjust the operating parameters of a drum dryer to obtain the desired properties (e.g. moisture content, fineness) of the solid HMO product. For instance, the drum-drying step may take place at a temperature of 100 to 160-170° C., more preferably at a temperature of 110-170° C., even more preferably at a temperature of 110-140° C. or 120-160° C., at atmospheric pressure. The heated drum usually rotates at a speed of from 0.3 rpm to several (i.e. 2-5 or even 9-10) rpm, providing a residence time of the aqueous composition on the surface of the heated drum of several seconds to 20-180 sec. Surprisingly, no significant degradation or coloration of the HMO was noticed running drum dryer at atmospheric pressure, with temperature above 100° C.

### Belt Drying, Vacuum Belt Drying

In belt dryers, products which are mostly wet are diffused equally on a wire mesh belt into the infeed chamber of the belt dryer. Most of the belt dryers have a wire mesh belt in a horizontal position which conveys the product through the drying sections. Gas or air flows circulate through the drying sections over the wet products. Each section within the dryer can be equipped with a heat exchanger or either a ventilating fan which enables the process operators to control each section separately. A belt dryer contains at least one entry point (infeed head), the conveyor belt to transport the product onto a porous wire mesh belt for air permeability and the discharge end for output. A belt dryer works with an air flow through or over the product to dry the product.

A vacuum belt dryer is a belt dryer that operates under vacuum. It comprises a belt upon which an aqueous composition is deposited, and where water is driven off using a combination of heat and low pressure. The belt is heated, typically by means of heating plates arranged above or below the belt which provide heat transfer via radiation.

The vacuum belt dryer used in the present technology may comprise various temperature zones, e.g. at least two, or at least three temperature zones. The operating temperature in each zone typically decreases from the first zone to the second and further zones.

Again, the person skilled in the art may adjust the operating parameters of a vacuum belt dryer, to obtain the desired properties. Vacuum belt drying may take place at a temperature of 90-160° C., preferably at a temperature of 110-140° C. (temperature of the plate), and at a pressure of 10-30 mbar. When vacuum belt drying, the aqueous composition typically has a residence time on the belt of 20-25 minutes. The feed flow of the aqueous composition to the vacuum belt dryer is typically between 150-180 g/min.

Belt drying, preferably vacuum belt drying, is a surprisingly effective industrial drying method that—in spite of the high temperature and the long residence time applied—does not provoke the thermal degradation of the HMOs and/or any other non-desirable change in the composition of HMOs. This is demonstrated in comparison tests with freeze drying which is known to be a gentle drying method (see examples).

### Granulation Followed by Drying

An overview of granulation technologies is found in Salman et al. Handbook of Powder Technology vol. 11, Granulation, Elsevier 2006. Granulation is the process of forming grains or granules from a powdery or solid substance, producing a granular material. Typically, granulation involves agglomeration of fine particles into larger granules, typically of size range between 0.2 and 4.0 mm depending on their subsequent use.

High shear granulators in general fall into two classes, namely horizontal axis and vertical axis, and can be either continuously operated or batch operated. High shear granulators use an impeller to vigorously agitate the powder and produce high-density granules. They are commonly found in the pharmaceutical, agrochemical and industries due to their ability to handle difficult feed formulations, including high viscosity binder fluids and fine cohesive powders. Impellers rotate at high speed (between 100 and 1500 rpm) on either a vertical or horizontal axis to create the agitation required for granulation. Typically, a secondary smaller impeller, called a chopper, is used. This rotates at much higher speeds (around 1500 rpm). Binder addition to high shear granulators can be in the form of a liquid spray or pouring.

Fluidized beds may also used be used for granulation. Fluidized-bed granulation in particular is very common, where atomizable liquids (e.g., suspensions, solutions, emulsions or melts) can be converted into free-flowing granular solids by integration of a number of processes like wetting, drying, size enlargement, shaping and homogenization or separation into a single step of the process chain by using high heat and mass transfer.

Another granulation process is extrusion-spheronisation (E-S). E-S is used to manufacture spherical or cylindrical pellets by extruding a semi-solid wet powder mass through a single die, a series of dies or a screen featuring many holes, then breaking up and rounding the extrudate on a rotating friction plate.

A rolling drum granulator is one of the most widely used granulating equipment devices, in which size enlargement is achieved by collisions in a bed of moist particles undergoing rolling motion. The rolling drum is the simplest continuous granulation device, and is widely used in the granulation of fertiliser and in the balling of iron ore.

Granulation can be carried out on an aqueous solution; in such cases, a solid dried carrier is required (e.g. solid HMO product from a previous batch). Suitably, the granulation step takes place at a temperature of 70-150° C. at atmospheric pressure; or a temperature of 30-100° C. under vacuum (50-100 mbars).

The granulation step and the drying step may be performed in the same apparatus, e.g. a fluidised-bed apparatus. The granulation process may therefore be fluidized-bed granulation or extrusion-spheronisation (E-S) granulation. Suitably, the granulation step is wet granulation.

### Solid HMO Product

Suitably, the solid HMO product of the invention comprises around 40% or more, such as around 50% or more, preferably around 60% or more by weight of synthetic HMOs, as a percentage of the total weight of the solid HMO product.

Some crystallisation of the synthetic HMOs could occur during the drying, due to the concentration step. The solid HMO product may therefore be amorphous solid HMO product, or crystalline solid HMO product, or a mixture of amorphous and crystalline solid HMO product.

### Additional Method Steps

The general methods provided herein may be supplemented by additional method steps, performed on the solid HMO product; i.e. post-drying.

In one aspect, the methods described herein further comprise the step of milling the solid HMO product to a solid HMO powder. Milling may provide a solid HMO powder with a various particle size distribution (D90) depending on the type of mill used e.g. impact mill, ball mill, forced sieve, or jet mill. As a further aspect, the methods may further comprise the step of sieving the solid HMO powder and separating the HMO powder into at least a first HMO powder fraction and a second HMO powder fraction. Sieving may provide HMO powder fractions with a particle size distribution (D90) depending the type of sieve used. The sieving or classification can be adapted to any customer requirement. From a food safety prospective a max. 1 mm sieving would be required, preferably 0.5-0.7 mm.

### Food Product

The solid HMO product may also be formulated in a food product, in particular a nutritional formulation, e.g. an infant nutritional formulation.

Nutritional formulations comprising solid HMO product may be foods, drinks or feeds. The nutritional formulation may contain edible micronutrients, vitamins and minerals as well. The amounts of such ingredients may vary depending on whether the formulation is intended for use with normal, healthy infants, children, adults or subjects having specialized needs (e.g. suffering from metabolic disorders). Micronutrients include for example edible oils, fats or fatty acids (such as coconut oil, soy-bean oil, monoglycerides, diglycerides, palm olein, sunflower oil, fish oil, linoleic acid, linolenic acid etc.), carbohydrates (such as glucose, fructose, sucrose, maltodextrin, starch, hydrolyzed cornstarch, etc.) and proteins from casein, soy-bean, whey or skim milk, or hydrolysates of these proteins, but protein from other source (either intact or hydrolysed) may be used as well. Vitamins may be chosen from the group consisting of vitamin A, B1, B2, B5, B6, B12, C, D, E, H, K, folic acid, inositol and nicotinic acid. The nutritional formulation may contain the following minerals and trace elements: Ca, P, K, Na, Cl, Mg, Mn, Fe, Cu, Zn, Se, Cr or I.

In a preferred embodiment the nutritional formulation is an infant nutritional formulation. Infant nutritional formulation means a foodstuff intended for particular nutritional use by infants during the first 4-6 months of life and satisfying by itself the nutritional requirements of infants. It may contain one or more probiotic Bifidobacterium species, prebiotics such as fructooligosaccharides and galactooligosaccharides, proteins from casein, soy-bean, whey or skim milk, carbohydrates such as lactose, saccharose, maltodextrin, starch or mixtures thereof, lipids (e.g. palm olein, sunflower oil, safflower oil) and vitamins and minerals essential in a daily diet. The infant formula contains solid HMO product in a total amount of 0.1-3.0 g/100 g formula.

In another preferred embodiment the nutritional formulation may be a food supplement comprising solid HMO product. The food supplement may comprise one or more probiotics in an amount sufficient to achieve the desired effect in an individual, preferably in children and adults. The food supplement may also contain vitamins, minerals, trace elements and other micronutrients as well. The food supplement may be for example in the form of tablets, capsules, pastilles or a liquid. The supplement may contain conventional additives selected from but not limited to binders, coatings, emulsifiers, solubilising agents, encapsulating agents, film forming agents, adsorbents, carriers, fillers, dispersing agents, wetting agents, jellifying agents, gel forming agents, etc. The daily dose of solid HMO product ranges from 0.1 to 3.0 g.

The following numbered aspects of the invention are provided:

Aspect 1. A method for providing a solid HMO product comprising one or more synthetic human milk oligosaccharides (HMOs), HMO precursors and/or by-products of HMO synthesis, said method comprising a step of drum-drying an aqueous composition comprising said one or more human milk oligosaccharides (HMOs), HMO precursors and/or by-products of the HMO synthesis.
Aspect 2. The method according to aspect 1, further comprising the step of milling the solid HMO product to a solid HMO powder.
Aspect 3. The method according to aspect 2, further comprising the step of sieving the solid HMO powder and separating the HMO powder into at least a first HMO powder fraction and a second HMO powder fraction.
Aspect 4. The method according to any one of the preceding aspects, wherein the solid HMO product comprises around 40% or more, such as around 50% or more, preferably around 60% or more by weight of synthetic HMOs, as a percentage of the total weight of the solid HMO product.
Aspect 5. The method according to any one of the preceding aspects, wherein the synthetic HMOs are selected from one or more of LNT, LNnT, 2′-FL, 3-FL, DFL, LNFP I, 3′-SL, 6′-SL, FSL, LST a, LST b, and DS-LNT, preferably LNT, LNnT, 2′-FL, 3-FL, DFL, LNFP I, 3′-SL and 6′-SL.
Aspect 6. The method according to any one of the preceding aspects, wherein the aqueous composition is an aqueous solution or an aqueous suspension of said one or more synthetic human milk oligosaccharides (HMOs), HMO precursors and/or by-products of HMO synthesis, preferably an aqueous solution of said one or more synthetic human milk oligosaccharides (HMOs), HMO precursors and/or by-products of HMO synthesis.
Aspect 7. The method according to any one of the preceding aspects, wherein the aqueous composition comprises two or more HMOs, such as e.g. three or more HMOs, or four or more HMOs.
Aspect 8. The method according to any one of the preceding aspects, wherein the aqueous composition comprises preferably 5 or more HMOs, from the group consisting of LNT, LNnT, Z-FL, 3-FL, DFL, LNFP I, 3′-SL, 6′-SL, FSL, LST a, LST b, and DS-LNT.
Aspect 9. The method according to any one of the preceding aspects, wherein the drum-drying step takes place at a temperature of 100-160° C., preferably at a temperature of 110-140° C., at atmospheric pressure.
Aspect 10. The method according to any one of the preceding aspects, wherein—in said drum-drying step—the aqueous composition has a residence time on the drum of 20-180 seconds.
Aspect 11. The method according to any one of the preceding aspects, further comprising the step of producing said one or more synthetic HMOs by means of fermentation, enzymatic reaction or synthetic reaction, prior to said step of drum-drying.
Aspect 12. The method according to aspect 11, wherein one or more purification steps are carried out on the HMO fermentation broth, enzymatic reaction milieu or synthetic reaction mixture, prior to said step of drum-drying.
Aspect 13. The method according to aspect 12, wherein said one or more purification steps carried out on the fermentation broth, enzymatic reaction milieu or synthetic reaction mixture are selected from one or more of:
removing solid material;
removing salts and charged molecules;
removing uncharged or non-charged material;
removing HMO precursors and/or by-products;
separating HMOs, if an HMO mixture is produced;
removing an excess of water.
   Aspect 14. The method according to any one of aspects 11-13, wherein said one or more synthetic HMOs are produced by means of fermentation and the fermentation broth is further processed to prepare an aqueous composition comprising said one or more synthetic human milk oligosaccharides (HMOs).
   Aspect 15. The method according to aspect 13 or aspect 14, wherein the method comprises:
a) clarifying the broth to remove suspended particulates and contaminants, particularly cells, cell components, insoluble metabolites and debris from a fermentation process; then
b) removing substantially all the proteins, as well as peptides, amino acids, RNA and DNA and any endotoxins and glycolipids that could interfere with the subsequent purification step, from the aqueous solution obtained in step a).
   Aspect 16. The method according to aspect 15, wherein the method comprises:
i) ultrafiltration (UF),
ii) nanofiltration (NF), and
iii) treatment with an ion exchange resin.
   Aspect 17. The method according to aspect 11, wherein the one or more synthetic HMOs are produced by means of fermentation, and wherein the fermentation broth is fed directly to the drum dryer as the aqueous composition comprising said one or more synthetic human milk oligosaccharides (HMOs), without any intermediate purification steps.
   Aspect 18. The method according to any one of the preceding aspects, wherein the drying step takes place to a moisture content in the solid HMO product of below 15%, preferably below 10%, more preferably below 7%, most preferably below 6%.
   Aspect 19. The method according to any one of the preceding aspects, wherein the drum-dryer used in the step of drum-drying does not comprise any applicator rollers.
   Aspect 20. The method according to any one of the preceding aspects, wherein the drum-dryer used in the step of drum-drying is a double drum dryer.
   Aspect 21. The method according to any one of aspects 1-19, wherein the drum-dryer used in the step of drum-drying is a vacuum drum dryer.
   Aspect 22. A drum-dried solid HMO product comprising one or more synthetic human milk oligosaccharides (HMOs), obtained by the method of any one of aspects 1-21.
   Aspect 23. A solid HMO product comprising one or more human milk oligosaccharides (HMOs), said solid HMO product having a glass transition temperature (T_{g}) higher than 120° C.
   Aspect 24. The solid HMO product according to any one of aspects 22-23, wherein the solid HMO product comprises one or more amorphous HMOs, or a mixture of amorphous and crystalline HMOs, or a crystalline HMO.
   Aspect 25. A food product, in particular a nutritional formulation, e.g. an infant nutritional formulation, comprising a solid HMO product according to any one of aspects 21-23.
   Aspect 26. A method for providing a solid HMO product comprising one or more synthetic human milk oligosaccharides (HMOs), HMO precursors and/or by-products of HMO synthesis, said method comprising a step of belt drying, preferably vacuum belt drying, an aqueous composition comprising said one or more human milk oligosaccharides (HMOs), HMO precursors and/or by-products of the HMO synthesis.
   Aspect 27. The method according to aspect 26, further comprising the step of milling the solid HMO product to a solid HMO powder.
   Aspect 28. The method according to aspect 27, further comprising the step of sieving the solid HMO powder and separating the HMO powder into at least a first HMO powder fraction and a second HMO powder fraction.
   Aspect 29. The method according to any one of aspects 26-28, wherein the solid HMO product comprises around 40% or more, such as around 50% or more, preferably around 60 or more by weight of synthetic HMOs, as a percentage of the total weight of the solid HMO product.
   Aspect 30. The method according to any one of aspects 26-29, wherein the synthetic HMOs are selected from one or more of LNT, LNnT, 2′-FL, 3-FL, DFL, LNFP I, 3′-SL, 6′-SL, FSL, LST a, LST b, and DS-LNT, preferably LNT, LNnT, 2′-FL, 3-FL, DFL, LNFP I, 3′-SL and 6′-SL.
   Aspect 31. The method according to any one of aspects 26-30, wherein the aqueous composition is an aqueous solution or an aqueous suspension of said one or more synthetic human milk oligosaccharides (HMOs), HMO precursors and/or by-products of HMO synthesis, preferably an aqueous solution of said one or more synthetic human milk oligosaccharides (HMOs), HMO precursors and/or by-products of HMO synthesis.
   Aspect 32. The method according to any one of aspects 26-31, wherein the aqueous composition comprises two or more HMOs, such as e.g. three or more HMOs, or four or more HMOs.
   Aspect 33. The method according to any one of aspects 26-32, wherein the aqueous composition comprises preferably 5 or more HMOs, from the group consisting of LNT, LNnT, Z-FL, 3-FL, DFL, LNFP I, 3′-SL, 6′-SL, FSL, LST a, LST b, and DS-LNT.
   Aspect 34. The method according to any one of aspects 26-33, wherein the vacuum belt dryer operates at a temperature of 90-160° C., preferably at a temperature of 110-140° C., and at a pressure of 10-30 mbar.
   Aspect 35. The method according to any one of aspects 26-34, further comprising the step of producing said one or more synthetic HMOs by means of fermentation, enzymatic reaction or synthetic reaction, prior to said step of belt drying, preferably vacuum belt drying.
   Aspect 36. The method according to aspect 35, wherein one or more purification steps are carried out on the HMO fermentation broth, enzymatic reaction milieu or synthetic reaction mixture, prior to said step of belt drying, preferably vacuum belt drying.
   Aspect 37. The method according to aspect 36, wherein said one or more purification steps carried out on the fermentation broth, enzymatic reaction milieu or synthetic reaction mixture are selected from one or more of:
removing solid material;
removing salts and charged molecules;
removing uncharged or non-charged material;
removing HMO precursors and/or by-products;
separating HMOs, if an HMO mixture is produced;
removing an excess of water.
   Aspect 38. The method according to any one of aspects 35-37, wherein said one or more synthetic HMOs are produced by means of fermentation and the fermentation broth is further processed to prepare an aqueous composition comprising said one or more synthetic human milk oligosaccharides (HMOs).
   Aspect 39. The method according to aspect 37 or aspect 38, wherein the method comprises:
a) clarifying the broth to remove suspended particulates and contaminants, particularly cells, cell components, insoluble metabolites and debris from a fermentation process; then
b) removing substantially all the proteins, as well as peptides, amino acids, RNA and DNA and any endotoxins and glycolipids that could interfere with the subsequent purification step, from the aqueous solution obtained in step a).
   Aspect 40. The method according to aspect 39, wherein the method comprises:
i) ultrafiltration (UF),
ii) nanofiltration (NF), and
iii) treatment with an ion exchange resin.
   Aspect 41. The method according to aspect 35, wherein the one or more synthetic HMOs are produced by means of fermentation, and wherein the fermentation broth, as the aqueous composition comprising said one or more synthetic human milk oligosaccharides (HMOs), is fed directly to the belt dryer, preferably vacuum belt dryer, without any intermediate purification steps.
   Aspect 42. The method according to any one of aspects 26-41, wherein the drying step takes place to a moisture content in the solid HMO product of below 15%, preferably below 10%, more preferably below 7%, most preferably below 6%.
   Aspect 43. The method according to any one of aspects 26-42, wherein the vacuum belt dryer comprises at least three temperature zones.
   Aspect 44. A belt dried, preferably vacuum belt dried solid HMO product comprising one or more synthetic human milk oligosaccharides (HMOs), obtained by the method of any one of aspects 26-43.
   Aspect 45. A solid HMO product comprising one or more human milk oligosaccharides (HMOs), said solid HMO product having a glass transition temperature (T_{g}) more than 100° C., such as from around 105° C. to around 160° C., e.g. around 100-150° C., around 120-140° C.
   Aspect 46. The solid HMO product according to any one of aspects 44-45, wherein the solid HMO product comprises one or more amorphous HMOs, or a mixture of amorphous and crystalline HMOs, or a crystalline HMO.
   Aspect 47. A food product, in particular a nutritional formulation, e.g. an infant nutritional formulation, comprising a solid HMO product according to any one of aspects 44-46.
   Aspect 48. A method for providing a solid HMO product comprising one or more synthetic human milk oligosaccharides (HMOs), HMO precursors and/or by-products of HMO synthesis, said method comprising a step of granulating an aqueous composition comprising said one or more human milk oligosaccharides (HMOs), HMO precursors and/or by-products of the HMO synthesis to form a granulated composition, followed by a step of drying said granulated composition.
   Aspect 49. The method according to aspect 48, wherein the granulation step is wet granulation.
   Aspect 50. The method according to any one of aspects 48-49, wherein the granulation step and the drying step are performed in the same apparatus.
   Aspect 51. The method according to any one of aspects 48-50, further comprising the step of milling the solid HMO product to a solid HMO powder.
   Aspect 52. The method according to aspect 51, further comprising the step of sieving the solid HMO powder and separating the HMO powder into at least a first HMO powder fraction and a second HMO powder fraction.
   Aspect 53. The method according to any one of aspects 48-52, wherein the solid HMO product comprises around 40% or more, such as around 50% or more, preferably around 60% or more by weight of synthetic HMOs, as a percentage of the total weight of the solid HMO product.
   Aspect 54. The method according to any one of aspects 48-53, wherein the synthetic HMOs are selected from one or more of LNT, LNnT, 2′-FL, 3-FL, DFL, LNFP I, 3′-SL, 6′-SL, FSL, LST a, LST b, and DS-LNT, preferably LNT, LNnT, 2′-FL, 3-FL, DFL, LNFP I, 3′-SL and 6′-SL.
   Aspect 55. The method according to any one of aspects 48-54, wherein the aqueous composition is an aqueous solution or an aqueous suspension of said one or more synthetic human milk oligosaccharides (HMOs), HMO precursors and/or by-products of HMO synthesis, preferably an aqueous solution of said one or more synthetic human milk oligosaccharides (HMOs), HMO precursors and/or by-products of HMO synthesis.
   Aspect 56. The method according to any one of aspects 48-55, wherein the aqueous composition comprises two or more HMOs, such as e.g. three or more HMOs, or four or more HMOs.
   Aspect 57. The method according to any one of aspects 48-56, wherein the aqueous composition comprises preferably 5 or more HMOs, from the group consisting of LNT, LNnT, Z-FL, 3-FL, DFL, LNFP I, 3′-SL, 6′-SL, FSL, LST a, LST b, and DS-LNT.
   Aspect 58. The method according to any one of aspects 48-57, wherein the granulation step takes place at a temperature of 70-150° C. at atmospheric pressure.
   Aspect 59. The method according to any one of aspects 48-58, wherein said granulation is fluidized-bed granulation or extrusion-spheronisation (E-S) granulation.
   Aspect 60. The method according to any one of aspects 48-59, further comprising the step of producing said one or more synthetic HMOs by means of fermentation, enzymatic reaction or synthetic reaction, prior to said step of granulation.
   Aspect 61. The method according to aspect 60, wherein one or more purification steps are carried out on the HMO fermentation broth, enzymatic reaction milieu or synthetic reaction mixture, prior to said step of granulation.
   Aspect 62. The method according to aspect 61, wherein said one or more purification steps carried out on the fermentation broth, enzymatic reaction milieu or synthetic reaction mixture are selected from one or more of:
removing solid material;
removing salts and charged molecules;
removing uncharged or non-charged material;
removing HMO precursors and/or by-products;
separating HMOs, if an HMO mixture is produced;
removing an excess of water.
   Aspect 63. The method according to any one of aspects 48-62, wherein said one or more synthetic HMOs are produced by means of fermentation and the fermentation broth is further processed to prepare an aqueous composition comprising said one or more synthetic human milk oligosaccharides (HMOs).
   Aspect 64. The method according to aspect 62 or aspect 63, wherein the method comprises:
a) clarifying the broth to remove suspended particulates and contaminants, particularly cells, cell components, insoluble metabolites and debris from a fermentation process; then
b) removing substantially all the proteins, as well as peptides, amino acids, RNA and DNA and any endotoxins and glycolipids that could interfere with the subsequent purification step, from the aqueous solution obtained in step a).
   Aspect 65. The method according to aspect 64, wherein the method comprises:
i) ultrafiltration (UF),
ii) nanofiltration (NF), and
iii) treatment with an ion exchange resin.
   Aspect 66. The method according to aspect 63, wherein the one or more synthetic HMOs are produced by means of fermentation, and wherein the fermentation broth is fed directly to the granulator as the aqueous composition comprising said one or more synthetic human milk oligosaccharides (HMOs), without any intermediate purification steps.
   Aspect 67. The method according to any one of aspects 48-66, wherein the granulation and drying steps take place to a moisture content in the solid HMO product of below 15%, preferably below 10%, more preferably below 7%, most preferably below 6%.
   Aspect 68. A granulated, dried solid HMO product comprising one or more synthetic human milk oligosaccharides (HMOs), obtained by the method of any one of aspects 48-67.
   Aspect 69. A solid HMO product comprising one or more human milk oligosaccharides (HMOs), said solid HMO product having a glass transition temperature (T_{g}) of around 120° C., or higher.
   Aspect 70. The solid HMO product according to any one of aspects 68-69, wherein the solid HMO product comprises one or more amorphous HMOs, or a mixture of amorphous and crystalline HMOs, or a crystalline HMO.
   Aspect 71. A food product, in particular a nutritional formulation, e.g. an infant nutritional formulation, comprising a solid HMO product according to any one of aspects 68-70.
   Aspect 72. A method for producing crystalline HMO in a predetermined polymorphic form from an aqueous composition of said HMO, said method comprising the steps of:
optionally, seeding said aqueous composition of said HMO with a seed crystal of said HMO in said predetermined polymorphic form, and
drying said aqueous composition to a water content of less than 10 w/w % thereby producing crystalline HMO in said predetermined polymorphic form.
   Aspect 73. The method according to aspect 72, wherein the aqueous composition of said HMO has a Brix of between 45 and 90, preferably 60 and 80, preferably about 75.
   Aspect 74. The method according to aspect 72 or aspect 73, wherein the drying is belt drying, preferably vacuum belt drying.
   Aspect 75. The method according to any one of aspects 72-74, wherein the HMO is 2′-FL.
   Aspect 76. The method according to any one of aspects 72-75, wherein the polymorphic form is polymorph II.
   Aspect 77. The method according to any one of aspects 72-76, wherein the aqueous composition is a fermentation broth from a fermentation process for producing said HMO.

The technology has been described with reference to a number of aspects and embodiments. These aspects and embodiments can be combined as desired by the skilled person, while remaining within the scope of the present invention, as defined by the appended claims. In particular, features relating to the methods described herein are also applicable to the solid HMO products, inasmuch as the method features give rise to identifiable differences in the solid HMO products themselves.

### EXAMPLES

### Vacuum Belt Drying

### Example 1

2′-FL-containing broth was generated by fermentation using a genetically modified E. coli strain of LacZ⁻, LacY⁺ phenotype, wherein said strain comprises a recombinant gene encoding an alpha-1,2-fucosyltransferase enzyme which is able to transfer fucose of GDP-fucose to the internalized lactose and genes encoding a biosynthetic pathway to GDP-fucose.

The fermentation was performed by culturing the strain in the presence of exogenously added lactose and a suitable carbon source, thereby producing 2′-FL which was accompanied with DFL and unreacted lactose as major carbohydrate impurities in the fermentation broth.

The broth was pH adjusted to pH 3.6 and subjected to ultrafiltration (15 kD, Kerasep Ceramic membranes). The UF permeate was further concentrated and diafiltrated by NF (Trisep UA 60 membranes) before being treated by an ion exchanger for demineralization and discoloration. The ion exchanger is a strongly acidic cation exchanger resin (Dowex 88) connected to a weakly basic anion exchanger resin (Dowex 66). The treated product was further decolorized by active charcoal (CPG-L F 20×40) before being concentrated by nanofiltration and vacuum evaporation. Product can be dried by spray drying or crystallized with the aid of acetic acid as described in WO 2016/095924. In the case of spray drying the final composition will include the carbohydrate impurities contained in the fermentation broth (lactose, DFL).

A 2′-FL solution (Brix 58) was prepared by dissolving spray dried/crystalline powder (ratio 2:1) at 60° C. The resulting solution was adjusted to pH 4.2 with NaOH. A part of the so-obtained solution was freeze dried whereas another part was dried on a vacuum belt dryer pilot unit (ZWAG, Switzerland, 20 mbars, heating zone 1 130° C., heating zone 2 120° C., heating zone 3 90° C., cooling zone 30° C., residence time 20 min, feed flow 150 g solution/min) to get a dried solid. The solid was further manually milled and sieved (1 mm) to give a white powder (residual humidity by Karl-Fisher: 6.0%).

Particle size distribution:

| | |
|---|---|
| D10 | 35 μm |
| D50 | 213 μm |
| D90 | 475 μm |

The XRPD spectrum (recorded on Philips model PW1710/PW1820 instrument, using Cu Kα radiation (λ=0.15418 nm) shows the sample is overwhelmingly (>95%) amorphous, containing a small amount of crystalline 2′-FL form II.

The freeze dried and the vacuum belt dried samples were analyzed by HPAE chromatography. The data showed that the concentrations of the respective ingredients practically did not differ in both samples (after normalization on dry matter). Specifically, concentrations of impurities that may be products of a possible thermal degradation and/or rearrangement process of 2′-FL (e.g. fucose, lactose, lactulose, fucosyl lactulose, galactose, glucose, fucosyl galactose, fructose) as well as concentrations of unspecified impurities, respectively, were practically the same in the samples. These data indicate that the harsher conditions of belt drying surprisingly does not change the quality of the obtainable solid 2′-FL product.

### Example 2

2′-FL-containing broth was generated by fermentation using a genetically modified E. coli strain of LacZ⁻, LacY⁺ phenotype, wherein said strain comprises a recombinant gene encoding an alpha-1,2-fucosyltransferase enzyme which is able to transfer fucose of GDP-fucose to the internalized lactose and genes encoding a biosynthetic pathway to GDP-fucose. The fermentation was performed by culturing the strain in the presence of exogenously added lactose and a suitable carbon source, thereby producing 2′-FL which was accompanied with DFL and unreacted lactose as major carbohydrate impurities in the fermentation broth.

The broth was pH adjusted to pH 3.6 and subjected to ultrafiltration (15 kD, Kerasep Ceramic membranes). The UF permeate was further concentrated and diafiltrated by NF (Trisep UA 60 membranes) before being treated by an ion exchanger for demineralization and discoloration. The ion exchanger is a strongly acidic cation exchanger resin (Dowex 88) connected to a weakly basic anion exchanger resin (Dowex 66). The treated product was further decolorized by active charcoal (CPG-L F 20×40) before being concentrated by nanofiltration and vacuum evaporation. Product can be dried by spray drying or crystallized with the aid of acetic acid as described in WO 2016/095924. In the case of spray drying the final composition will include the carbohydrate impurities contained in the fermentation broth (lactose, DFL).

A supersaturated 2′-FL solution (Brix 74) was prepared by dissolving spray dried/crystalline powder (ratio 3:1) at 90° C. The resulting solution was cooled to 70° C. and adjusted to pH 4.2 with NaOH. A part of the so-obtained solution was freeze dried whereas another part was dried on a vacuum belt dryer pilot unit (ZWAG, Switzerland, 20 mbars, heating zone 1 130° C., heating zone 2 120° C., heating zone 3 90° C., cooling zone 30° C., residence time 20 min, feed flow 150 g solution/min) to get a dried solid. The solid was further manually milled and sieved (1 mm) to give a white powder (residual humidity residual humidity by Karl-Fisher: 4.3%).

DSC analysis: no glass transition temperature between 0 and 120° C.

Particle size distribution:

| | |
|---|---|
| D10 | 33 μm |
| D50 | 228 μm |
| D90 | 514 μm |

The freeze dried and the vacuum belt dried samples were analyzed by HPAE chromatography. The data showed that the concentrations of the respective ingredients practically did not differ in both samples (after normalization on dry matter). Specifically, concentrations of impurities that may be products of a possible thermal degradation and/or rearrangement process of 2′-FL (e.g. fucose, lactose, lactulose, fucosyl lactulose, galactose, glucose, fucosyl galactose, fructose) as well as concentrations of unspecified impurities, respectively, were practically the same in the samples. These data indicate that the harsher conditions of belt drying surprisingly does not change the quality of the obtainable solid 2′-FL product.

### Example 3

2′-FL-containing broth was generated by fermentation using a genetically modified E. coli strain of LacZ−, LacY+ phenotype, wherein said strain comprises a recombinant gene encoding an alpha-1,2-fucosyltransferase enzyme which is able to transfer fucose of GDP-fucose to the internalized lactose and genes encoding a biosynthetic pathway to GDP-fucose. The fermentation was performed by culturing the strain in the presence of exogenously added lactose and a suitable carbon source, thereby producing 2′-FL which was accompanied with DFL and unreacted lactose as major carbohydrate impurities in the fermentation broth.

The broth was pH adjusted to pH 3.6 and subjected to ultrafiltration (15 kD, Kerasep Ceramic membranes). The UF permeate was further concentrated and diafiltrated by NF (Trisep UA 60 membranes) before being treated by an ion exchanger for demineralization and discoloration. The ion exchanger is a strongly acidic cation exchanger (Dowex 88) connected to a weakly basic anion exchanger (Dowex 66). The treated product was further decolorized by active charcoal (CPG-L F 20×40) before being concentrated by nanofiltration and vacuum evaporation to brix 76. The solution was seeded (2′-FL seed polymorph II) and cooled down to room temperature for 2 days.

The obtained suspension was rewarmed to 50° C. and was dried on a vacuum belt dryer pilot unit (ZWAG, Switzerland, 20 mbars, heating zone 1 130° C., heating zone 2 120° C., heating zone 3 90° C., cooling zone 30° C., residence time 20 min, feed flow 150 g solution/min) to get a dried solid. The solid was further manually milled and sieved (1 mm) to give a white powder (residual humidity residual humidity by Karl-Fisher: 2.5%)

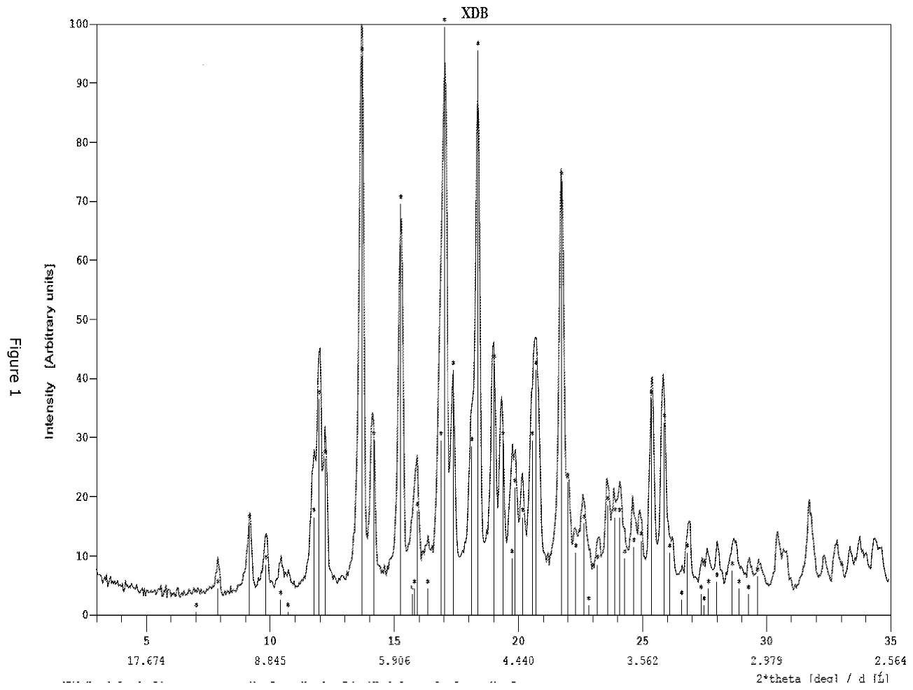

The XRPD spectrum (recorded on Philips model PW1710/PW1820 instrument, using Cu Kα radiation (λ=0.15418 nm) shows the sample is well crystalline 2′-FL form II (see FIG. 1; the peaks with asterisk * belong to reference 2′-FL polymorph II that is described in WO 2011/150939).

Particle size distribution:

| | |
|---|---|
| D10 | 63 μm |
| D50 | 364 μm |
| D90 | 601 μm |

1. A process for providing a solid human milk oligosaccharide (HMO) product comprising one or more synthetic HMOs, HMO precursors, or by-products of HMO synthesis, said process comprising a step of
belt drying,
drum-drying, or
granulating
an aqueous composition comprising said one or more HMOs, HMO precursors, or by-products of the HMO synthesis.

2. The process according to claim 1, wherein the aqueous composition comprises one or more HMOs and either HMO precursors, by-products of HMO synthesis, or both.

3. The process according to claim 1, wherein the belt drying comprises vacuum belt drying.

4. The process according to claim 1, wherein the granulating the aqueous composition to form a granulated composition is followed by a step of drying said granulated composition.

5. The process according to claim 1, further comprising the step of milling the solid HMO product to a solid HMO powder.

6. The process according to claim 5, further comprising the step of sieving the solid HMO powder and separating the HMO powder into at least a first HMO powder fraction and a second HMO powder fraction.

7. The process according to claim 1, wherein the solid HMO product comprises around 40% or more by weight of synthetic HMOs, as a percentage of the total weight of the solid HMO product.

8. The process according to claim 1, wherein the one or more synthetic HMOs are selected from the group consisting of LNT, LNnT, 2′-FL, 3-FL, DFL, LNFP I, 3′-SL, 6′-SL, FSL, LST a, LST b and DS-LNT; or a combination thereof.

9. The process according to claim 1, wherein the aqueous composition is an aqueous solution or an aqueous suspension of said one or more synthetic HMOs, HMO precursors, or by-products of HMO synthesis.

10. The process according to claim 1, wherein the drying step takes place to a moisture content in the solid HMO product of below 15%

11. The process according to claim 1, further comprising the step of producing said one or more synthetic HMOs by means of fermentation, enzymatic reaction or synthetic reaction, prior to said step of drum drying, belt drying, or granulation.

12. The process according to claim 11, wherein one or more purification steps are carried out on the HMO fermentation broth, enzymatic reaction milieu or synthetic reaction mixture, prior to said step of drum drying, belt drying, or granulation.

13. The process according to claim 12, wherein said one or more purification steps carried out on the fermentation broth, enzymatic reaction milieu or synthetic reaction mixture are selected from the group consisting of:
removing solid material;
removing salts and charged molecules;
removing uncharged or non-charged material;
removing HMO precursors and/or by-products;
separating HMOs, if an HMO mixture is produced; and
removing an excess of water;
or a combination thereof.

14. The process according to claim 1, wherein said one or more synthetic HMOs are produced by means of fermentation and the fermentation broth is further processed to prepare an aqueous composition comprising said one or more synthetic human milk oligosaccharides (HMOs).

15. The process according to claim 13, wherein the method comprises:
a) clarifying the broth to remove suspended particulates and contaminants, from a fermentation process to form an aqueous solution; then
b) removing substantially all the proteins, as well as peptides, amino acids, RNA and DNA and any endotoxins and glycolipids that could interfere with the subsequent purification step, from the aqueous solution obtained in step a).

16. The process according to claim 15, wherein the method comprises:
i) ultrafiltration (UF),
ii) nanofiltration (NF), and
iii) treatment with an ion exchange resin.

17. The process according to claim 11, wherein the one or more synthetic HMOs are produced by means of fermentation, and wherein the fermentation broth, as the aqueous composition comprising said one or more synthetic human milk oligosaccharides (HMOs), is fed directly to the drum dryer, belt dryer, or granulator, without any intermediate purification steps.

18. The process according to claim 1, wherein the solid HMO product comprises one or more amorphous HMOs, or a mixture of amorphous and crystalline HMOs, or a crystalline HMO.

19. (canceled)

20. A belt dried solid HMO product comprising one or more synthetic human milk oligosaccharides (HMOs), obtained by the process of claim 1.

21. (canceled)

22. A solid HMO product comprising one or more human milk oligosaccharides (HMOs), said solid HMO product having a glass transition temperature (T_{g}) around 120° C., or higher.

23. A food product comprising a solid HMO product according to claim 20.

24.-29. (canceled)
